(19) United States
(12) Patent Application Publication
(10) Pub. No.: US 2023/0131712 A1
(43) Pub. Date: Apr. 27, 2023

(54) BLOOD VOLUME MEASUREMENT WITH FLUORESCENT DYE

(71) Applicant: DAXOR CORP., New York, NY (US)

(72) Inventors: Jonathan Feldschuh, New York, NY (US); Atilio Anzellotti, Oak Ridge, TN (US); Nancy Tommye Jordan, Knoxville, TN (US); Boyce Lee Muller, Knoxville, TN (US); Robin D. Zimmer, Loudon, TN (US); Adam Michael Cable, Lenoir City, TN (US)

(73) Assignee: DAXOR CORP., New York, NY (US)

(21) Appl. No.: 17/612,273

(22) PCT Filed: Apr. 29, 2021
(86) PCT No.: PCT/US2021/029930

§ 371 Date: Nov. 18, 2021

Related U.S. Application Data
(60) Provisional application No. 63/017,799, filed on Apr. 30, 2020.

(51) Int. Cl. G01N 33/58; G01N 33/543; A61B 5/0275

(52) U.S. Cl. CPC G01N 33/582; A61B 5/0275; G01N 33/54388

(57) ABSTRACT

Systems and methods are provided for measuring blood volume of a living being using fluorescent dye. The present invention greatly simplifies the performance of a blood volume measurement by utilizing a novel fluorescent tracer. An injection and sampling kit for the performance of an indicator dilution measurement, comprising a) a labelled fluorescent injectate, and b) a plurality of collection cassettes, and a calibration kit comprising a plurality of calibrated standard cassettes of identical conformation to the cassettes in b), corresponding to known dilutions of the injectate (a).

### CROSS-REFERENCE TO RELATED APPLICATIONS

This application claims the benefit of U.S. Provisional Pat. Application No. 63/017,799, filed on Apr. 30, 2020, the contents of which are herein incorporated by reference into the subject application.

### FIELD OF THE INVENTION (TECHNICAL FIELD

The present invention relates to systems and methods for analyzing blood of a living being.

### BACKGROUND

A blood volume analyzer (BVA) is an instrument or system capable of measuring and reporting the volume of blood of a living being. The Daxor BVA-100 Blood Volume Analyzer, based on U.S. Pat. No. 5,024,231 is a commercially available, FDA-cleared device. It operates on the indicator-dilution principle. I-131-labelled Human Serum Albumin (HSA) is injected into a subject’s blood stream, and various samples of blood are taken at timed intervals after mixing has occurred. The use of radiation for the detection mechanism results in requirements for radioactive materials licensing that many facilities (such as physicians’ offices, outpatient clinics, dialysis centers, etc.) do not possess. These factors limit widespread practical clinical use of the blood volume measurement, which has been proven in numerous published clinical studies to have significant health benefits in guiding treatment.

### SUMMARY OF THE INVENTION

Methods and systems are presented for analyzing the blood of a living being using fluorescent tracers.

### BRIEF SUMMARY OF EMBODIMENTS OF THE PRESENT INVENTION

The present invention greatly simplifies the performance of a blood volume measurement by utilizing a novel fluorescent tracer, the composition of which is the subject of another patent application. The novel fluorescent in ternary complex has the virtue of being stable in the bloodstream, permitting accurate indicator dilution measurements. The present invention will also operate with any other fluorescent tracer. This removes cumbersome radiation safety procedures. The present invention also has the advantage of using less blood in the performance of a test, as fluorescent detection generally requires a smaller volume of blood than radiation detection. For a typical test performed with the BVA-100, 42 ml of patient blood might be required (6 patient samples, plus a background sample, each 6 ml). The current invention might use quantities between 10 µl and 500 µl per sample, for a reduction of total blood required of orders of magnitude.

In one embodiment, the analyzer is a handheld device with an integrated touchscreen, counting well, and barcode reader, which performs measurements of the concentration of a fluorescent tracer present in samples of whole blood introduced into a fluorescent counting chamber, as well as hematocrit (Hct) values, so as to determine blood volume and associated values.

In one embodiment, all counting is done using cassettes of identical conformation.

In one embodiment, these cassettes are membrane-based.

In another embodiment, a cassette with a full-wicking membrane is used to measure the fluorescent concentration, and the analyzer reads the fluorescence level from the entire surface area of the membrane visible through an opening in the cassette.

In another embodiment, the cassettes use lateral flow methodology, whereby a monoclonal antibody to the fluorescent tracer is applied to a line across the membrane, and readings are made from the area of said line.

In another embodiment, the measurements of fluorescence are performed using an integrated device with a touchscreen for input and display of results, and a receptacle for the introduction of cassettes to be measured.

In another embodiment, an integrated optical detection system is used to quantify the level of fluorescence, using defined frequencies of excitation and emission corresponding to the properties of the florescent tracer.

In another embodiment, the device includes a barcode reader to input the dilution volumes for each cassette and ensure that the injectate lot id and standard lot id match.

In another embodiment, the cassette includes a second membrane that is capable of making a simultaneous hematocrit determination.

In another embodiment, a kit is provided containing materials needed to perform a blood volume measurement using the system. An example of such a kit is an injection and sampling kit for the performance of an indicator dilution measurement, comprising a labelled fluorescent injectate, and a plurality of collection cassettes. Another example of such a kit is a calibration kit, comprising a plurality of calibrated standard cassettes of identical conformation to the cassettes in the injection and sampling kit, corresponding to known dilutions of the injectate from a given lot. Another example of such a kit is a full measurement kit, combining the contents of the two previously described kits.

### BRIEF DESCRIPTION OF THE SEVERAL VIEWS OF THE DRAWINGS

FIG. 1A shows a sample measurement cassette using the full-wicking principle, with the cover (below) removed from the base (above). Patient samples are placed in the “T” pad, as well as in the circular hole; quality control (QC) fluid is placed in the “QC” pad. The T and QC samples wick to the entire surface. The Hct sample wicks linearly.

FIG. 1B shows a detail of the membrane below the “T” and “QC” pads, showing the absorbent membrane of precise size affixed to a non-absorbent backing pad.

FIG. 2A shows a sample measurement cassette using the lateral flow principle, with the cover (below) removed from the base (above). Patient samples are placed in the two circular holes.

FIG. 2B shows a detail of the membrane below the “BV” pads, showing the lines where material is affixed to indicate the presence of test material.

FIG. 3 shows a cassette (301) about to be inserted into the carrier tray (302) which contains an insert (303) to accommodate the cassette and facilitate precise insertion and easy removal of the cassette.

FIG. 4 shows a rendering of an instrument capable of performing a blood volume measurement via reading of a cassette.

FIG. 5 shows a table with the contents of three kits for use in performing a complete blood volume measurement.

FIG. 6 shows the contents of a full measurement kit for performing a complete blood volume measurement from FIG. 5.

FIG. 7 shows the kit from FIG. 6 in a packaging tray.

FIG. 8 shows a system for analyzing the blood of a living subject, using fluorescent tracer and cassettes of matched conformation for sample measurement and calibration.

### DETAILED DESCRIPTION OF THE INVENTION

A system is provided for automatically analyzing blood of a living subject, comprising a plurality of concentration-measuring cassettes, a plurality of calibration cassettes containing known concentrations of fluorescent activity, a reader capable of making readings of fluorescence levels, a user interface operatively connected to the reader and configured for entry and display of information, one or more processors operatively coupled to a memory and configured to execute programmed instructions stored in the memory to carry out a method comprising the steps of:
a. measuring a sample of whole blood from the subject in a counting cassette to determine a background level of fluorescence;
b. injecting the subject with a precise, known volume of fluorescent tracer;
c. measuring the level of fluorescence in a plurality of calibration cassettes matched to the batch of fluorescent tracer used in step b) and creating a calibration curve relating fluorescence to volume of dilution therefrom;
d. at one or more timed intervals after the injection, placing a sample of whole blood from the subject in a counting cassette and measuring a post-injection level of fluorescence;
e. quantifying the volume of dilution of each sample from steps a) and d) using interpolation between the measured activity of the calibration cassettes via in step c) and the respective volume of dilution corresponding to their known concentrations;
f. calculating, by the one or more processors, a blood volume (BV), plasma volume (PV), and red cell volume (RCV) for the subject;
g. calculating, by the one or more processors, an ideal blood volume (iBV), ideal plasma volume (iPV), and red cell volume (iRCV) for the subject based on subject descriptive data such as height, weight, and gender; and
h. displaying, by the one or more processors, at the user interface, the results.

The calculations in steps e), f) and g) can be performed as follows, for example. Volume of dilution can be measured by fluorescence in a subject sample compared with the measured fluorescence detected in a calibration standard cassette of known volume of dilution. The activity can be measured directly in the fluorescence reader, and using the reference standards (representing dilution volumes such as 1000 ml, 2000 ml, 4000 ml, 6000 ml, and 8000 ml) the activity can be translated into a subject volume of dilution via interpolation, so long as the activity falls into the range encompassed by the standards. Suitable standards can be employed based on the approximate expected unknown volume.

The overall whole body Hct (oHct) is related to the peripheral Hct by the following relationship:

$$o H c t = p H c t ∗ p a f$$

where

$$p a f = .9009$$

This is due to the fact that blood cells are more concentrated in the peripheral circulation (from which blood samples are drawn) than the average value for the whole body; the constant paf is derived as the product 0.99 * 0.91, as described in patent 5,024,231, or a similar constant value. Red Cell Volume and Plasma volume are related to Blood Volume as follows:

$$B V = P V + R C V$$

$$R C V = B V ∗ o H c t = B V ∗ p H c t ∗ p a f$$

$$P V = B V ∗ \left(1 - o H c t\right) = B V ∗ \left(1 - p H c t ∗ p a f\right)$$

When the tracer is known to leak out of the bloodstream (as e.g. labelled albumin transudates at a rate of approximately 0.25%/min), a time-zero BV can be calculated using a log-linear regression of BV values obtained at various points.

The Ideal Hct (iHct) is defined to be:

$$\begin{matrix}i H c t ≡ \left\{\begin{matrix}0.45 f o r M a l e s \\ 0.40 f o r F e m a l e s\end{matrix}\right) \\ i H c t ≡ \left\{\begin{matrix}0.45 f o r M a l e s \\ 0.40 f o r F e m a l e s\end{matrix}\right)\end{matrix}$$

The Ideal Red Cell Volume (iRCV) and Ideal Plasma Volume (iPV) are calculated from the iBV. Note that the iHct is a peripheral Hct value, so the peripheral adjustment factor is required:

$$i B V = i P V + i R C V$$

$$i R C V = i B V ∗ i H c t ∗ p a f$$

$$i P V = i B V - i R C V = i B V ∗ \left(1 - i H c t ∗ p a f\right)$$

Several approaches to incorporating Hct information into the blood volume measurement are possible. Recent publications show promise of hematocrit determinations using filter paper. It is known that hematocrits of higher values move slower on cellulose strips than low-values hematocrits. A set of hematocrit standards are made to use in the quantification of unknown hematocrits be compared to centrifuged hematocrits, considered to be the Gold Standard method for hematocrit. Acoustofluidics (the use of sound vibration to separate suspended particles in a mixture) can be used both to estimate particle density (and hence estimate Hct), as well as to provide clear visual access to plasma to facilitate fluorescent detection. There is also the option of manual entry of hematocrit for the blood sample from independent conventional methods, and to receive this data electronically from laboratory information systems.

Immunochromatograph materials available for dipstick or lateral flow tests are either glass, cellulose, cotton or nitrocellulose fibers. In dipstick assays, cellulose, cotton or glass typically have acceptable volume retention. These membranes are hydrophobic and may be synthetic polyester, cotton, or microfiber glass with a neutral pH allowing the whole blood sample to stay within the within sample pad while the plasma migrates down the test strip. In some uses, cellulose membrane has shown higher volume retention than glass.

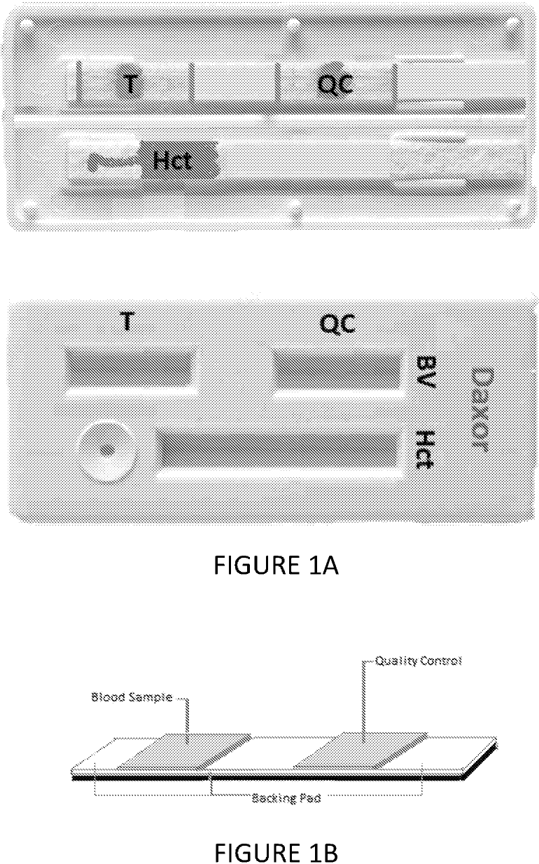

FIG. 1 shows a membrane-based cassette capable of measuring fluorescence and Hct simultaneously when inserted into a suitable reader. The cassette operates on the full-wicking principle: fluid applied to the membrane will wick to cover the surface uniformly, but not spread beyond the surface. A membrane pad with strong retention of blood sample is applied to a backing strip approximately 5 mm wide x 25 mm long and a second membrane pad about 18 mm distance from blood sample pad that is 5 x 20 mm in size. This strip is housed in a plastic cassette with open windows approximately 4 mm by 18-20 mm at each membrane pad.

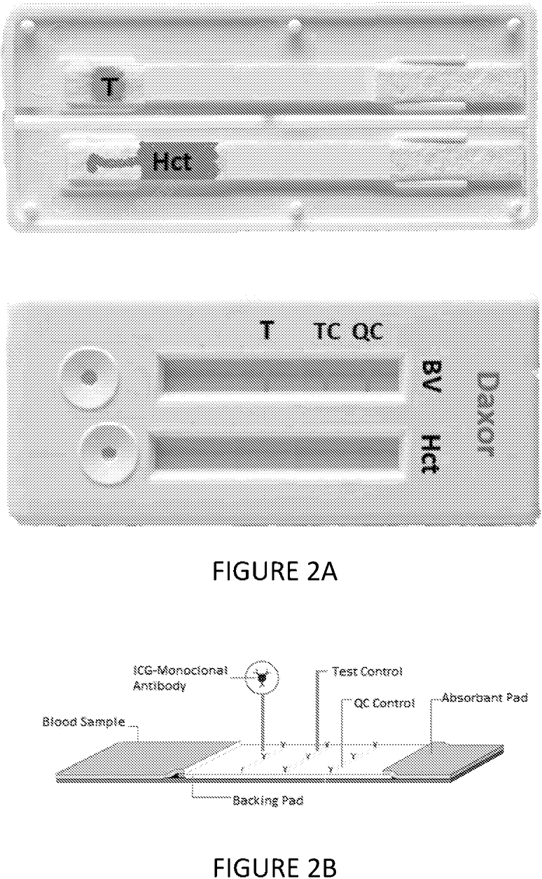

The measurement principle for the membrane can use an aggregate fluorescent reading from the entire membrane surface (since the fluid spreads evenly). Alternatively, as shown in FIG. 2B, via the use of monoclonal antibodies to the fluorescent tracer, e.g. Indocyanine Green (ICG), the fluorescence can be concentrated along a fixed line of the cassette for improved measurement resolution and accuracy.

A backing membrane approximately 5 mm wide x 65 mm long is used to build up the lateral flow membranes so that a capillary flow of the test sample moves through the selected sample pad onto the nitrocellulose where it comes in contact with the ICG-monoclonal line at ‘T’ (FIGS. 2A, 2B). At this time, all the injectate in the patient’s blood plasma sample will bind to the ICG-monoclonal ‘T’-line and produce a fluorescent signal in proportion to the amount of ICG injectate in that test sample. The non-ICG components of the plasma sample continues its capillary travel to the TC line, QC Line, and finally to the absorbent pad of test strip (FIG. 2B).

The cassette of FIGS. 1A-1B can be employed for measuring blood volume by following a method comprising the following steps:
a. To the area marked “T” on the plastic cassette (FIG. 1), add 10 ul of whole blood, using the disposable pipette included in the Testing Kit.
b. To the area marked “QC” on the plastic cassette (FIG. 1), add 10 ul of Quality Control Sample, using the disposable pipette included in the Testing Kit.
c. Insert the Cassette into the Fluorescent Blood Volume Reader, and then slide it into the Reader. The reader has an opening where the cassette is inserted into a drawer which has a bed to precisely locate the cassette windows in the reader.
d. The fluorescent calibration curve for every lot is supplied with each lot of cassettes used in the Testing Kit on a card that is read by the Reader.
e. The drawer is then slid into the Fluorescent Blood Volume Reader.
f. The signal intensity on the strip is analyzed. The Reader excites the blood sample on the test strip membrane with flashes of precise duration of microseconds and intensity of a near-infrared wavelength of 780 nm focused beam. This Excitation beam causes the fluorescent tracer (FT) in the patient blood sample to fluoresce, with a peak intensity at 820 nm. The Reader measures the amount of fluorescent emission over a precise time duration in microseconds, and uses the calibration data for that cassette to calculate the volume of FT in the plasma contained in the whole blood sample. It then uses the indicator solution method to calculate the whole blood volume in the patient.
g. The results are shown on Reader screen.

The Lateral Flow cassette of FIGS. 2A-2B can be employed for measuring blood volume by following a method comprising the following steps:
h. To the area marked “T” on the plastic cassette (FIG. 1), add 10 ul of whole blood, using the disposable pipette included in Testing Kit.
i. To this same circular well marked ‘T’, add 75 ul of Kit Buffer with from the Test Buffer bottle. One drop from the Test Buffer bottle equals 25 uL (add 3 drops only to the circular well). This buffer starts the capillary push of the plasma from the whole blood sample down the test strip. The content of the buffer also contains components for the development of the TC and QC lines on the membrane. The TC Line shows the lateral flow system is properly working. The Fluorescent Reader will use the QC line to calculate a known value of the control, showing accuracy of results from test strip. Insert the DipStick Cassette into the Fluorescent Blood Volume Reader, and then slide it into the Reader. The reader has an opening where the cassette is inserted into a drawer which has a bed to precisely locate the cassette windows in the reader.
j. As soon as the TC line is apparent, the Lateral Flow Cassette is inserted into the Fluorescent Reader cassette drawer, and then slid into the Reader.
k. The signal intensity on the strip is analyzed. The Reader excites the blood sample on the test strip membrane with flashes of precise duration of microseconds and intensity of a near-infrared wavelength of 780 nm focused beam. This Excitation beam causes the fluorescent tracer (FT) in the patient blood sample to fluoresce, with a peak intensity at 820 nm. The Reader measures the amount of fluorescent emission over a precise time duration in microseconds, and uses the calibration data for that lot of FT injectate to calculate the volume of FT in the plasma contained in the whole blood sample. It then uses the indicator solution method to calculate the whole blood volume in the patient.
1. The results are shown on reader screen.

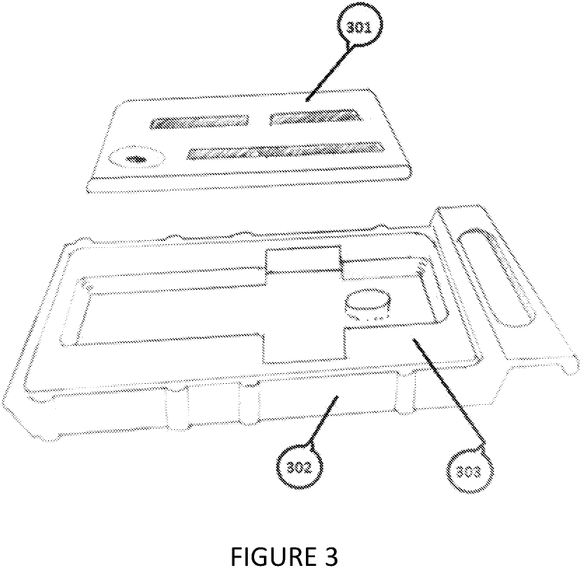

FIG. 3 shows a cassette (301) about to be inserted into the blood sample test drawer (302) which contains a cassette fixture insert (303) to accommodate the cassette and facilitate precise insertion and easy removal of the cassette. Both the One-Step Membrane fluorescence method of measuring whole body blood volumes, the Lateral Flow fluorescence method of measuring whole body blood volumes, and the reading of patient hematocrit in the same blood samples, are designed to utilize a common drawer system. Blood samples for the Lateral Flow or the One-Step test will use small flat plastic test cassettes which snap together like a clamshell, with the test strips inside. A cassette may contain one or two strips: a Lateral Flow test strip, a One-Step test strip, a hematocrit test, or a hematocrit test combined with a Lateral Flow or a One-Step test in the same cassette.

Each cassette (301) has a matching fixture (302) which fits snugly inside the test drawer (303). The cassette snaps into its matching fixture to position the cassette precisely and repeatedly, test to test, for the reading apparatus inside the Fluorescent Blood Volume Reader. Each cassette will have a bar code which the Reader uses to verify the type of test being run and to configure the instrument accordingly. In addition, each cassette bar code corresponds to a Lot Number of the Fluorescent Tracer injectate. A card which accompanies each cassette bears a 2-dimensional barcode which is read by the instrument to calibrate it to the Cassette Lot Number and the ICG-injectate of the Test Cassette being used.

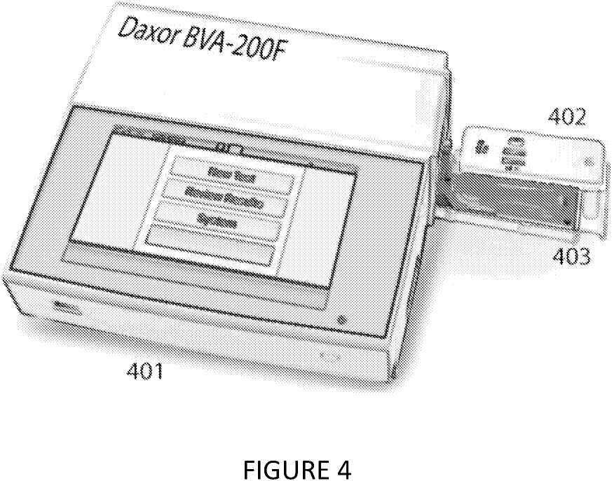

FIG. 4 shows a rendering of an instrument capable of performing a blood volume measurement via reading of a cassette. The device (401) has a touchscreen capable of receiving input and displaying results. A tray (403) allows the insertion of cassettes (402).

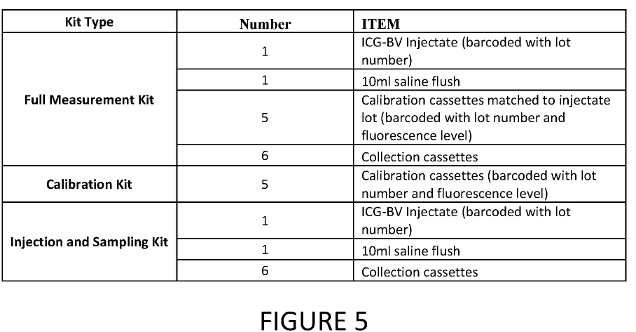

FIG. 5 shows a table listing the contents of various kits for performing a complete blood volume measurement. The Injection and Sampling Kit contains the fluorescent injectate, which has a barcoded identification of the lot number. The injectate is provided in a single use container, which is used in conjunction with a saline flush to ensure that the entire dose is delivered accurately and precisely to the subject. The kit also includes six collection cassettes: one for background (pre-injection) and five for post-injection samples. The Calibration Kit contains a set of 5 calibration cassettes matched to a specific injectate lot; each cassette is barcoded with lot number and fluorescence level. Each cassette contains printed information on the volume of dilution. A set for use in human adults might include the volumes 1000, 2000, 4000, 6000, and 8000 ml. For example, the “VOD: 1000 ml” calibration cassette would contain an amount of fluorescence equivalent to the matched injectate diluted into 1000 ml. A set of calibration cassettes for use in smaller or larger subjects might have respectively smaller or larger volumes of dilution. The calibration and collection cassettes are of identical conformation, so that samples and standards can be read in the same counter. Cassettes are labeled with human-readable printing and machine-readable barcode information. The Full Measurement Kit includes the contents of both the Injection and Sampling Kit and the Calibration Kit. One skilled in the art would recognize that other combinations of these kits might be desirable (e.g. a kit including quantity one of the Calibration Kit and quantity five of the Injection and Sampling Kit, for performing five separate blood volume measurements.)

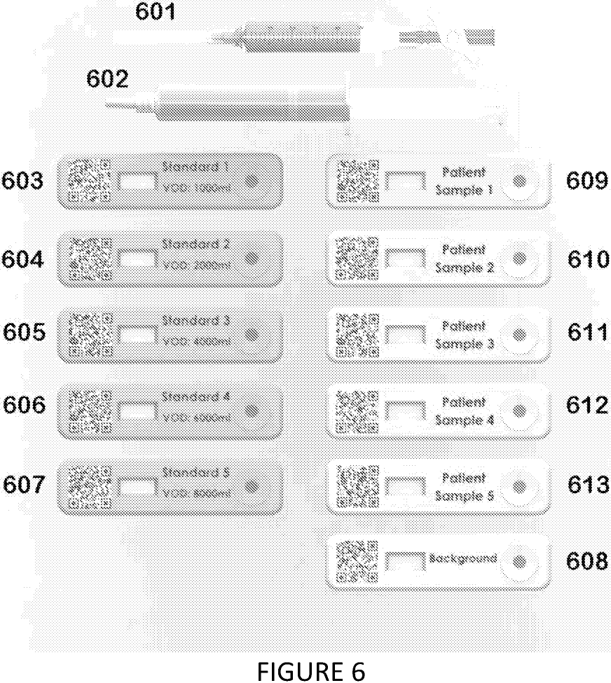

FIG. 6 shows the contents of an embodiment of the Full Measurement Kit from FIG. 5. These include: a single-use syringe containing injectate (601); a pre-filled flush syringe (602) containing a sufficient amount of sterile saline (e.g. 10 ml) to ensure that the injectate (601) is completely delivered to the subject; A set of standard cassettes (603-607) is provided to cover the expected range of volume of dilution to be encountered, in this case 1000-8000 ml; a sample collection cassette (608) for measurement of any background fluorescence that may be present in the subject prior to injection; and a set of patient sample collection cassettes (609-613) for collecting timed post-injection samples. All of the cassettes (603-613) have the same dimensions, such that they can be read in the same counter; they also feature both barcode labelling and printing to assist in the efficient performance of the test.

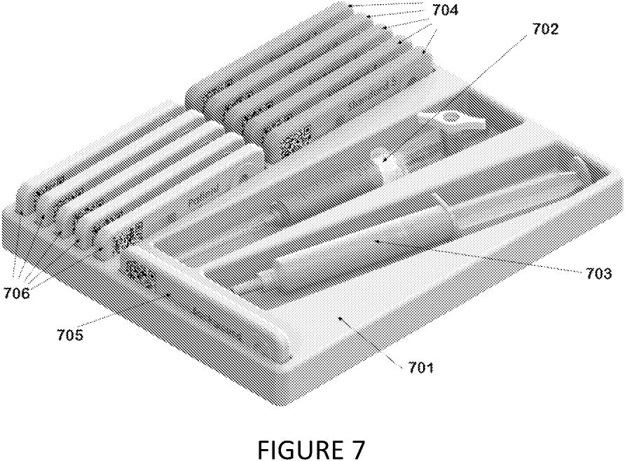

FIG. 7 shows an embodiment of the Full Measurement Kit from FIG. 5 as it might be packaged. All of the components are present in a molded tray (701), with a single-use syringe containing injectate (702); a pre-filled flush syringe (703); A set of standard cassettes (704); a sample collection background cassette (705); and a set of patient sample collection cassettes (706) for collecting timed post-injection samples.

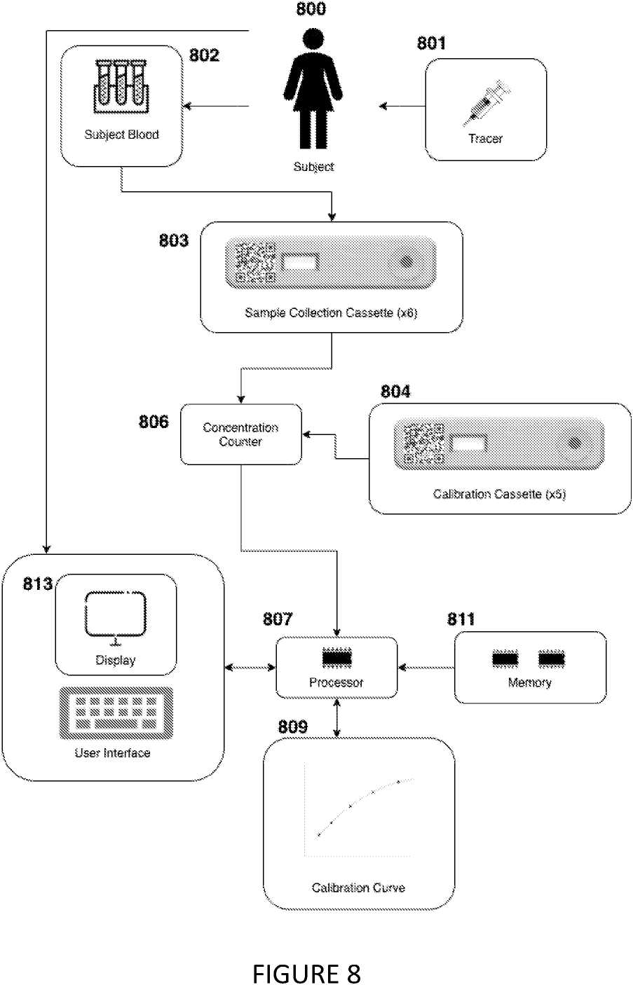

FIG. 8 shows an embodiment of a system for automatically analyzing blood of a living subject (800). Samples of subject blood (802) are collected, one background sample before the injection of a fluorescent tracer (801) into the subject (800), and a plurality (e.g. 5) at timed intervals after the injection. Each of the samples is placed in a sample collection cassette (803) and counted in a concentration counter (806) capable of quantifying fluorescent activity. A plurality (e.g. 5) of calibration cassettes (804) containing known concentrations of fluorescent activity, of the same conformation as the collection cassettes (803) are also measured in the counter (806). The counter (806) is connected to one or more processors (807) operatively coupled to a memory (811) and a user interface configured for entry and display of information (813). The processor (807) is configured to execute programmed instructions stored in the memory (811). The processor (807) constructs a calibration curve (809) from the readings of the calibration cassettes (804), and then determines the volume of dilution for each timed sample based on the net activity (sample less background) for each timed sample. The processor (807) calculates a time-zero blood volume and displays the results on the user interface (813).

Also provided is a method of performing the indicator dilution method to determine the blood volume of a subject, whereby
a) a sample of whole blood from the subject is placed into a counting cassette;
b) the subject is injected with the fluorescent injectate;
c) at one or more timed intervals after the injection, a sample of whole blood from the subject is placed into a counting cassette;
d) the level of fluorescence of each subject and standard cassette is determined using a counter capable of quantifying fluorescence;
e) the volume of dilution of each sample (from steps a and c) is determined using interpolation between the measured activity of the standards and their known volumes; and
f) blood volume (BV), plasma volume (PV), and red cell volume (RCV) are calculated for the subject.

The cassettes in this method can be membrane-based. They can use the full-wicking or lateral-flow methodology, as described above. The cassettes can also include an aperture for the application of subject blood for the measurement of Hct, in conjunction with the calculations in step f).

What is claimed is:

1. A system for automatically analyzing blood of a living subject, comprising a plurality of concentration-measuring cassettes, a plurality of calibration cassettes containing known concentrations of fluorescent activity, a reader capable of making readings of fluorescence levels, a user interface operatively connected to the reader and configured for entry and display of information, one or more processors operatively coupled to a memory and configured to execute programmed instructions stored in the memory to carry out a method comprising the steps of:
a) measuring a sample of whole blood from the subject in a counting cassette to determine a background level of fluorescence;
b) injecting the subject with a precise, known volume of fluorescent tracer;
c) measuring the level of fluorescence in a plurality of calibration cassettes matched to the batch of fluorescent tracer used in step b) and creating a calibration curve relating fluorescence to volume of dilution therefrom;
d) at one or more timed intervals after the injection, placing a sample of whole blood from the subject in a counting cassette and measuring a post-injection level of fluorescence;
e) quantifying the volume of dilution of each sample from steps a) and d) using interpolation between the measured activity of the calibration cassettes via in step c) and the respective volume of dilution corresponding to their known concentrations;
f) calculating, by the one or more processors, a blood volume (BV), plasma volume (PV), and red cell volume (RCV) for the subject;
g) calculating, by the one or more processors, an ideal blood volume (iBV), ideal plasma volume (iPV), and red cell volume (iRCV) for the subject based on subject descriptive data such as height, weight, and gender; and
h) displaying, by the one or more processors, at the user interface, the results.

2. The system of claim 1, where the sample collection cassettes include a separate receptacle for a blood sample to be used in determination of Hematocrit (Hct) by the system, for use in the calculations in steps f) and g).

3. The system of claim 1, where the cassettes are membrane-based.

4. The system of claim 3, where the cassettes use the full-wicking principle, and the analyzer reads the fluorescence level from the entire surface area of the membrane visible through an opening in the cassette.

5. The system of claim 3, where the cassettes use lateral flow methodology, whereby a monoclonal antibody to the fluorescent tracer is applied to a line across the membrane, and readings are made from the area of said line.

6. The system of claim 1, where the measurements of fluorescence are performed using an integrated device with a touchscreen for input and display of results, and a receptacle for the introduction of cassettes to be measured.

7. The system of claim 6, where the cassettes are measured using an optical system with defined frequencies of excitation and emission for fluorescent quantification.

8. The system of claim 6, where the device includes a barcode reader to input the dilution volumes for each cassette and ensure that the injectate lot id and standard lot id match.

9. An injection and sampling kit for the performance of an indicator dilution measurement, comprising
a) a labelled fluorescent injectate, and
b) a plurality of collection cassettes.

10. A calibration kit for the performance of an indicator dilution measurement in conjunction with the kit of claim 9, comprising a plurality of calibrated standard cassettes of identical conformation to the cassettes in b), corresponding to known dilutions of the injectate (a).

11. A full measurement kit for the performance of an indicator dilution measurement, comprising
a) a labelled fluorescent injectate,
b) a plurality of collection cassettes, and
c) a plurality of calibrated standard cassettes of identical conformation to b), corresponding to known dilutions of the injectate a).

12. A method of performing the indicator dilution method to determine the blood volume of a subject using the contents of the kit of claim 11, whereby
a) a sample of whole blood from the subject is placed into a counting cassette;
b) the subject is injected with the fluorescent injectate;
c) at one or more timed intervals after the injection, a sample of whole blood from the subject is placed into a counting cassette;
d) the level of fluorescence of each subject and standard cassette is determined using a counter capable of quantifying fluorescence;
e) the volume of dilution of each sample (from steps a and c) is determined using interpolation between the measured activity of the standards and their known volumes; and
f) blood volume (BV), plasma volume (PV), and red cell volume (RCV) are calculated for the subject.

13. The method of claim 12, where the counting cassettes contain an aperture for the application of subject blood for the measurement of Hct, in conjunction with the calculations in step f).

14. The method of claim 12, where the cassettes are membrane-based.

15. The method of claim 14, where the cassettes use the full-wicking principle, and the counter reads the fluorescence level from the entire surface are of the membrane visible through an opening in the cassette.

16. The method of claim 14, where the cassettes use lateral flow methodology, whereby a monoclonal antibody to the fluorescent tracer is applied to a line across the membrane, and readings are made from the area of said line.
